(19) United States
(12) Patent Application Publication
(10) Pub. No.: US 2025/0228748 A1
(43) Pub. Date: Jul. 17, 2025

(54) NASOJEJUNAL FEEDING TUBE DEVICES AND MATERIALS FOR RETENTION

(71) Applicant: BOSTON SCIENTIFIC SCIMED, INC., Maple Grove, MN (US)

(72) Inventors: Rosangel Aimee RAMOS ESPINOZA, Boston, MA (US); Nathan Thomas CUMMINGS, Worcester, MA (US); Laura Elizabeth CHRISTAKIS, Framingham, MA (US); Ryan V. WALES, Northborough, MA (US); John B. GOLDEN, Norton, MA (US); Jeff GRAY, Sudbury, MA (US); John Thomas FAVREAU, Charlton, MA (US); Paul SMITH, Smithfield, RI (US)

(21) Appl. No.: 19/016,198

(22) Filed: Jan. 10, 2025

Related U.S. Application Data
(60) Provisional application No. 63/621,876, filed on Jan. 17, 2024.

(51) Int. Cl. A61J 15/00

(52) U.S. Cl. CPC A61J 15/0023; A61J 15/0049; A61J 15/0069

(57) ABSTRACT

A device includes a flexible catheter and a material. The catheter includes at least a first channel extending therethrough and is sized and shaped to extend from a proximal end outside a human body to a distal end that can be guided to a target location within a gastrointestinal (GI) system. A first distal portion of the catheter includes at least one exit port communicating with the first channel for administering a fluid to a patient anatomy. The material is fixed to an exterior of the catheter or ejectable from an interior of the catheter into the location that, in a first state or shape, is suitable for guiding the catheter to the location and, in a second state or shape, is suitable for engaging an inner wall of the location to anchor the catheter in the location while administering the fluid via the first channel.

### PRIORITY CLAIM

The present disclosure claims priority to U.S. Provisional Patent Application Ser. No. 63/621,876 filed Jan. 17, 2024; the disclosure of which is incorporated herewith by reference.

### FIELD

present disclosure relates to tubes and/or catheters with retention features for anchoring in the small bowel.

### BACKGROUND

Pyloric stenosis constitutes a narrowing of the gastric outlet (pylorus) causing difficulties in passing food from the stomach into the small bowel. This can lead to a gastric outlet obstruction (stricture). The inability to pass food into the small bowel can reduce or completely impede a person's ability to digest food and absorb nutrients. There are several solutions to this problem, including placing a nasojejunal (NJ) feeding tube in the small bowel, e.g., jejunum. Another solution includes the creation of a surgical gastro-jejunostomy bypass bridging the stomach and the jejunum to bypass the area of the stricture. NJ tubes can be used for assisting endoscopic ultrasound guided gastroenterostomy (EUS-GE) procedures by instilling contrast or another fluid in the jejunum. In one example, contrast is provided via the NJ tube to help make visible a target to an operating physician, e.g., for a procedure including piercing the stomach and the jejunum and placing a stent (e.g., an AXIOS™ stent) therebetween to bypass the pylorus.

One issue with NJ tubes is that the tube may drift out of position during use so that fluids discharged by the tube are provided to an unintended location (i.e., as the outlet of the tube is no longer located in a target location). In one example, the NJ tube may migrate proximally during use. In feeding tube applications, this proximal drift may cause nutrients to be provided further upstream in the small bowel, or in the stomach proximal to the stricture, which would eliminate the benefits of the tube and may cause issues with digestion. In EUS-GE applications, the drift of the NJ tube can cause fluids (e.g., contrast fluid) to be provided to a location spaced from an intended target area. The stents for bypassing the pylorus may be mis-deployed if the physician is confused or misdirected due to the contrast fluid being provided to an unintended location during gastro-jejunostomy procedures.

### SUMMARY

The present disclosure relates to a device for administering a fluid to a patient anatomy. The device includes a flexible catheter including at least a first channel extending therethrough. The catheter is sized and shaped to extend from a proximal end outside a human body to a distal end that can be guided to a target location within a gastrointestinal (GI) system. A first distal portion of the catheter includes one or more exit ports communicating with the first channel for administering a fluid. In addition, the device includes a material fixed to an exterior of the catheter or ejectable from an interior of the catheter into the target location that, in a first state or shape, is suitable for guiding the catheter to the target location and, in a second state or shape, is suitable for engaging an inner wall of the target location to anchor the catheter in the target location while administering the fluid via the first channel.

In an embodiment, the catheter includes a second distal portion including one or more exit ports communicating with a second channel, the second distal portion distal to the first distal portion, the material comprising a fibrous gel or a fibrous glue injected through the second channel. In the first state or shape, the fibrous gel or the fibrous glue behaves as a gel or fluid for traversing the second channel. The fibrous gel or the fibrous glue is discharged out of the exit ports of the second distal portion and transitions into the second state or shape in which the fibrous gel or the fibrous glue solidifies into a fibrous bundle for engaging the inner wall of the target location.

In an embodiment, the fibrous gel or the fibrous glue is activated to transition into the fibrous bundle by fluid or gas in the GI system after discharge into the target location.

In an embodiment, the fibrous glue includes components that are mixed upon discharge into a target volume to transition into the fibrous bundle.

In an embodiment, the catheter includes a second distal portion at which the material is applied, the material comprising an adhesive, wherein, in the first state or shape, the adhesive behaves as a solid without adhesive qualities, and wherein the adhesive transitions into the second state or shape and behaves with the adhesive qualities after a duration suitable for guiding the catheter to the target location.

In an embodiment, the adhesive comprises a time-activated material with a selected intubation time for transitioning into the second state.

In an embodiment, the second distal portion of the catheter includes longitudinal indentations for applying the adhesive.

In an embodiment, the catheter includes a second distal portion at which the material is attached, the material comprising a flexible super-absorbent material, wherein, in the first state or shape, the material is contracted around the second distal portion. The material absorbs a further fluid to transition into the second state or shape in which the material expands.

In an embodiment, in the second state or shape, the material is of a size sufficient to prevent the material from passing proximally through a stricture in the GI system.

In an embodiment, the catheter includes a second distal portion at which the material is attached and further includes suction ports, the catheter including a second channel extending therethrough communicating with the suction ports, the material comprising a sponge. In the first state or shape, the sponge is fully contracted around the second distal portion. In the second state or shape, the sponge is released from the first state or shape and expands to engage the inner wall of the target location. In a third state or shape, a suction force is applied via the suction ports to distribute the suction force throughout the sponge to partially contract the sponge and the inner wall of the target location.

In an embodiment, the device further includes a sheath disposed around the second distal portion of the catheter, the sheath being translatable relative to the catheter from a first position to a second position, wherein the sponge is constrained by the sheath around the catheter in the first state or shape when the sheath is in the first position and translating the sheath into the second position unconstrains the sponge to allow the sponge to assume the second state.

In an embodiment, the device further includes a balloon adjacent to the distal end of the catheter, the catheter including a third channel extending therethrough communicating with an interior of the balloon so that, in a first state, the balloon is deflated and assumes a first shape suitable for guiding the distal end of the catheter to a further target location distal to the target location and, in a second state, the balloon is inflated and assumes a second shape in which the balloon extends radially from the catheter for contacting an inner wall of the further target location to anchor the catheter at the further target location. The first distal portion including the exit ports is distal to the sponge and proximal to the balloon.

In an embodiment, the sponge is formed from or incorporates an auxetic material that expands radially under tension or compression to transition the sponge into the second state or shape.

In an embodiment, the device further includes a clip housed within a cavity at the distal end in the first state, the clip being deployable from the distal end so that, in the second state, the clip grasps the inner wall of the target location.

In an embodiment, the clip is deployable by a coil and remains fixed to the catheter via a tether.

In addition, the present disclosure relates to a method for administering a fluid to a patient anatomy. The method includes guiding a flexible catheter to a target location within a gastrointestinal (GI) system, the catheter including at least a first channel extending therethrough, the catheter being sized and shaped to extend from a proximal end outside a human body to a distal end, a first distal portion of the catheter including one or more exit ports communicating with the first channel for administering a fluid; transitioning a material from a first state or shape to a second state or shape, the material being fixed to an exterior of the catheter or ejectable from an interior of the catheter into the target location, the first state or shape being suitable for guiding the catheter to the target location and the second state or shape being suitable for engaging an inner wall of the target location to anchor the catheter in the target location; and administering the fluid via the first channel.

In an embodiment, the catheter includes a second distal portion including one or more exit ports communicating with a second channel, the second distal portion distal to the first distal portion, the material comprising a fibrous gel or a fibrous glue injected through the second channel. In the first state or shape, the fibrous gel or the fibrous glue behaves as a gel or fluid for traversing the second channel. The fibrous gel or the fibrous glue is discharged out of the exit ports of the second distal portion and transitions into the second state or shape in which the fibrous gel or the fibrous glue solidifies into a fibrous bundle for engaging the inner wall of the target location.

In an embodiment, the catheter includes a second distal portion at which the material is applied, the material comprising an adhesive. In the first state or shape, the adhesive behaves as a solid without adhesive qualities. The adhesive transitions into the second state or shape and behaves with the adhesive qualities after a duration suitable for guiding the catheter to the target location.

In an embodiment, the catheter includes a second distal portion at which the material is attached, the material comprising a flexible super-absorbent material. In the first state or shape, the material is contracted around the second distal portion. The material absorbs a further fluid to transition into the second state or shape in which the material expands.

In an embodiment, the catheter includes a second distal portion at which the material is attached and further includes suction ports, the catheter including a second channel extending therethrough communicating with the suction ports, the material comprising a sponge. In the first state or shape, the sponge is fully contracted around the second distal portion. In the second state or shape, the sponge is released from the first state or shape and expands to engage the inner wall of the target location. In a third state or shape, a suction force is applied via the suction ports to distribute the suction force throughout the sponge to partially contract the sponge and the inner wall of the target location.

### BRIEF DESCRIPTION

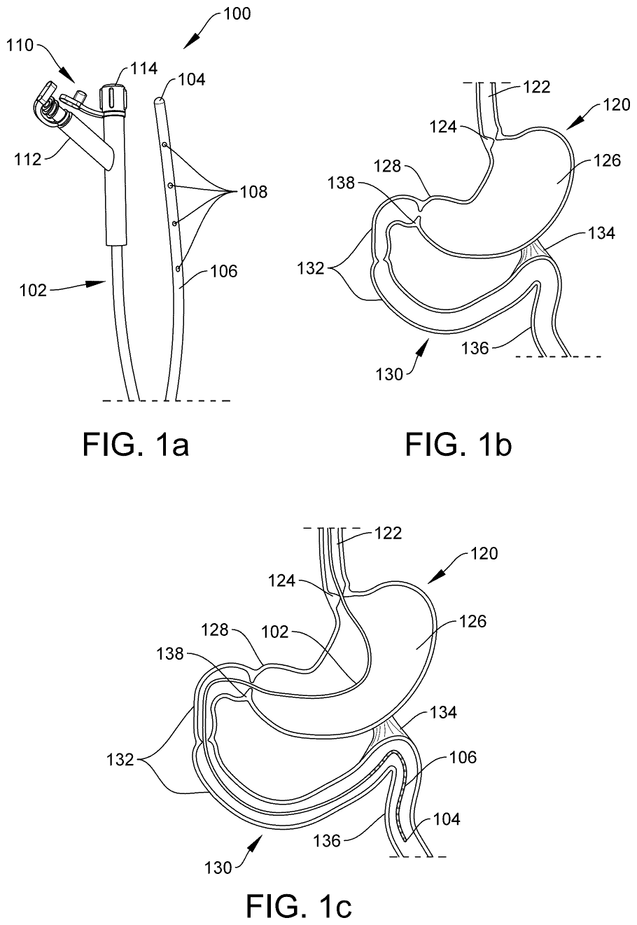

FIG. 1a shows a nasojejunal (NJ) tube device according to one example.

FIG. 1b shows a patient anatomy prior to inserting a NJ tube according to one example.

FIG. 1c shows the catheter of the NJ tube device of FIG. 1a placed in the patient anatomy of FIG. 1b according to one example.

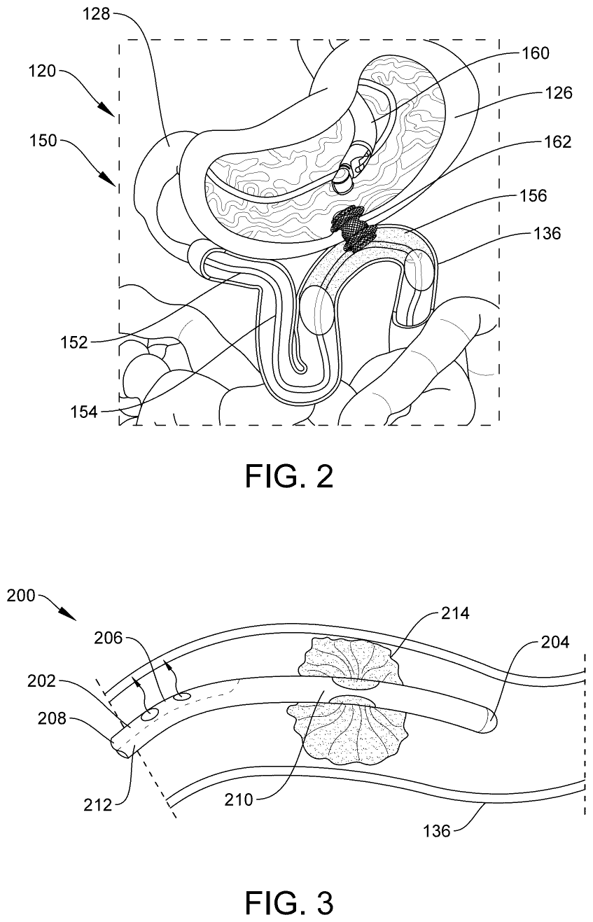

FIG. 2 shows a EUS-GE system for placing a gastro-jejunal stent in the anatomy of FIG. 1b according to one example.

FIG. 3 shows a catheter device including a fibrous bundle formed from a fibrous gel or glue discharged from a catheter within the patient anatomy of FIG. 1b according to a first example of these exemplary embodiments.

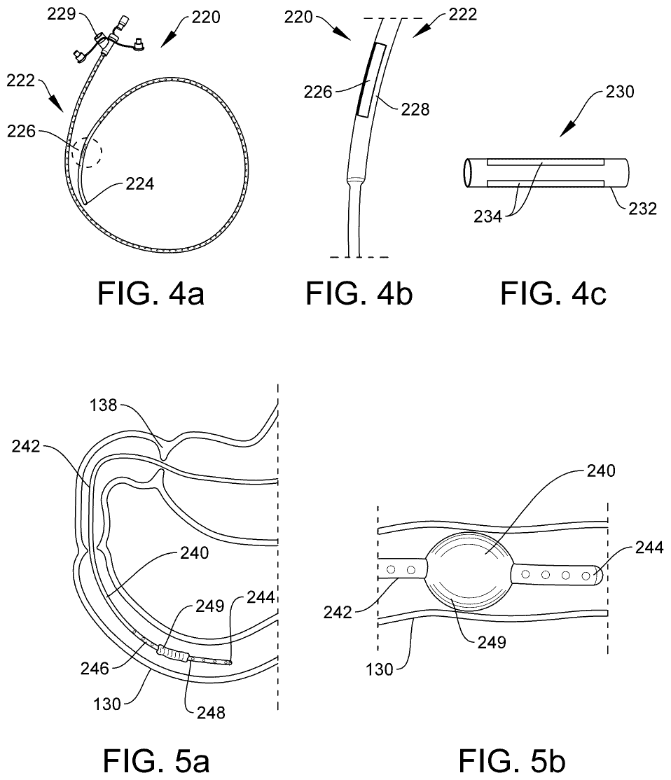

FIG. 4a shows a catheter device including a catheter with a second distal portion on which an adhesive is applied according to a second example of these exemplary embodiments.

FIG. 4b shows the adhesive of the catheter device of FIG. 4a according to the second exemplary embodiment.

FIG. 4c shows a second distal portion of a catheter device including indentations for adhesive along the second distal portion according to a third example of these exemplary embodiments.

FIG. 5a shows a catheter device including a superabsorbent expanding material attached to a catheter in a contracted state (first shape) within the patient anatomy of FIG. 1b according to a fourth example of these exemplary embodiments.

FIG. 5b shows the material of the catheter device in an expanded state (second shape) within the patient anatomy of FIG. 1b according to the fourth exemplary embodiment.

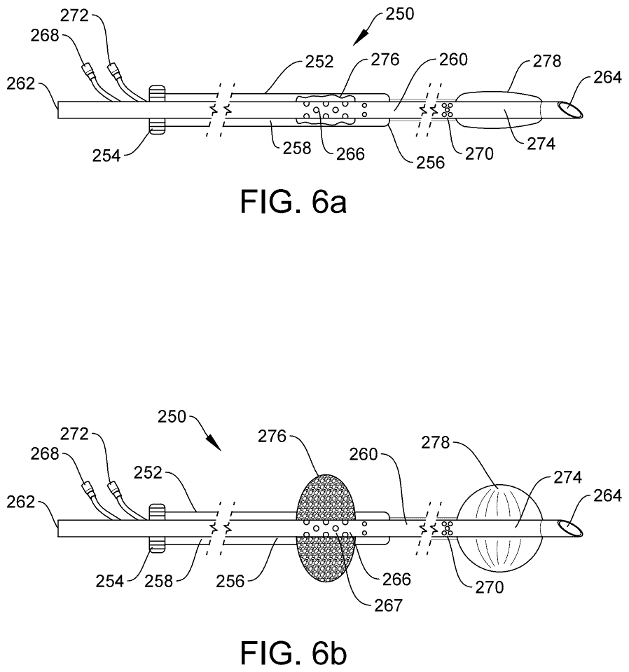

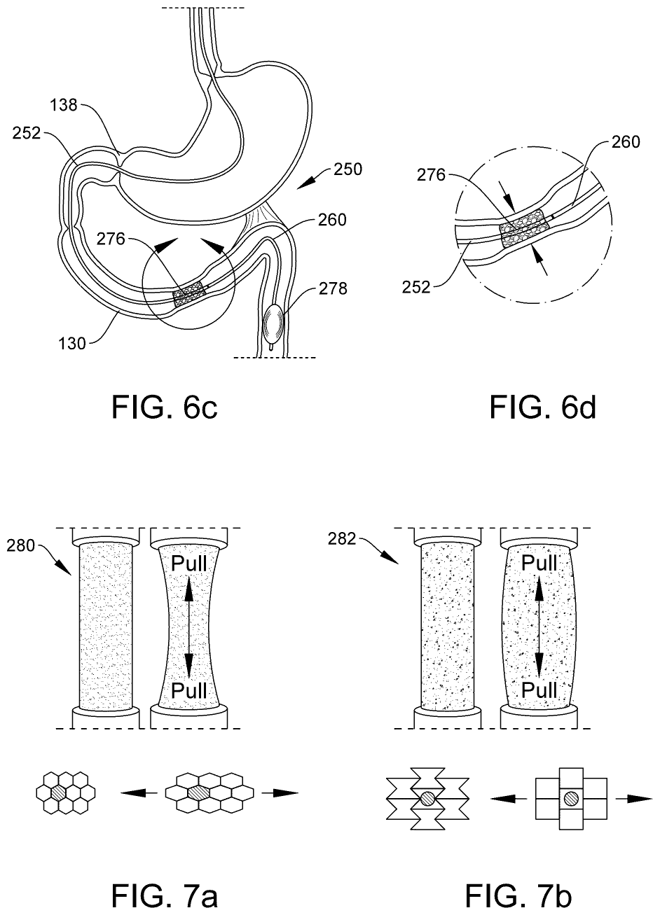

FIG. 6a shows a catheter device including a sponge for distributing a suction force in a fully contracted state (first shape) according to a fifth example of these exemplary embodiments.

FIG. 6b shows the catheter device of FIG. 6a with the sponge in an expanded state (second shape) according to the fifth exemplary embodiment.

FIG. 6c shows the catheter device of FIG. 6a with the sponge in a partially contracted state (third shape) under the suction force within the patient anatomy of FIG. 1b according to the fifth exemplary embodiment.

FIG. 6d shows the sponge in the partially contracted state (third shape) within the patient anatomy of FIG. 1b according to the fifth exemplary embodiment.

FIG. 7a shows a conventional material according to one example.

FIG. 7b shows an auxetic material according to a seventh example of these exemplary embodiments.

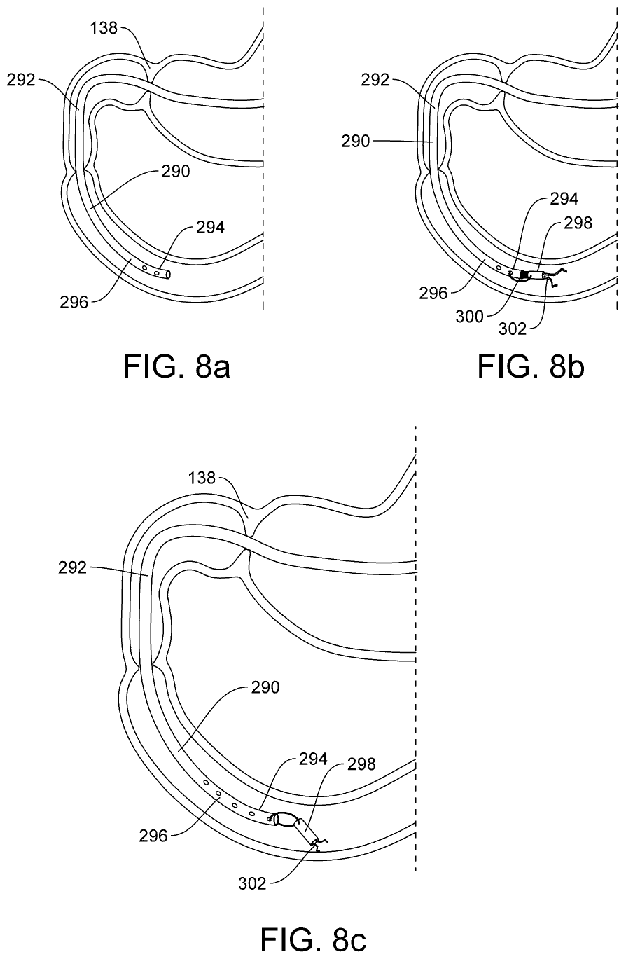

FIG. 8a shows a catheter device including a clip deployable from a distal end of a catheter in a withdrawn state within the distal end within the patient anatomy of FIG. 1b according to an eighth example of these exemplary embodiments.

FIG. 8b shows the catheter device of FIG. 8a with the clip in a partially deployed state within the patient anatomy of FIG. 1b according to the eighth exemplary embodiment.

FIG. 8c shows the catheter device of FIG. 8a with the clip in a fully deployed state engaging the inner wall of the small bowel within the patient anatomy of FIG. 1b according to the eighth exemplary embodiment.

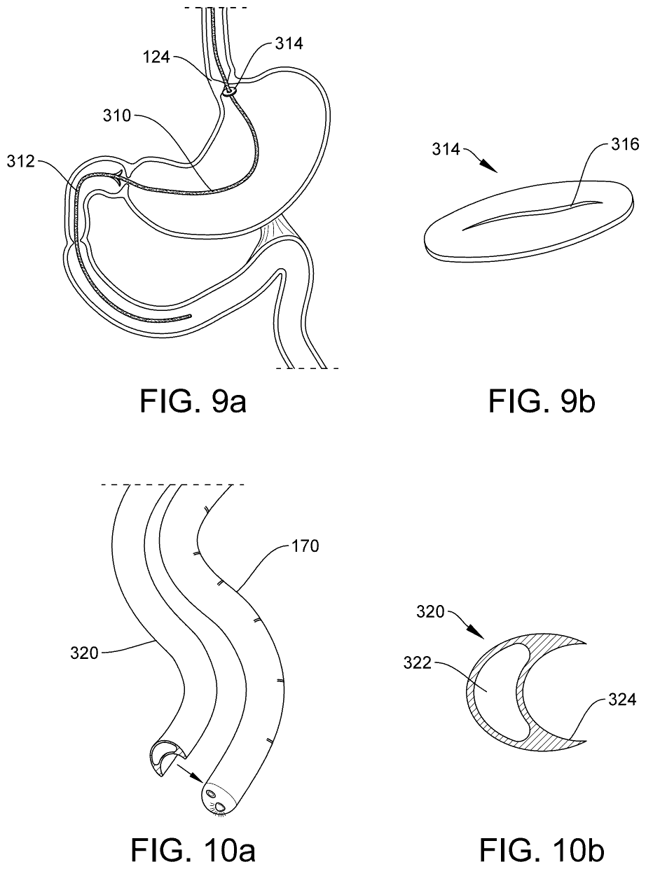

FIG. 9a shows a catheter device including a disk disposed around a catheter within the patient anatomy of FIG. 1b adjacent to the esophageal sphincter according to a ninth example of these exemplary embodiments.

FIG. 9b shows the disk of the catheter device of FIG. 9a according to the ninth exemplary embodiment.

FIG. 10a shows an accessory device for an endoscope comprising a channel through which a catheter can be passed according to a tenth example of these exemplary embodiments.

FIG. 10b shows a cross section of the accessory device of FIG. 10a according to the tenth exemplary embodiment.

### DETAILED DESCRIPTION

The present disclosure may be further understood with reference to the following description and the appended drawings, wherein like elements are referred to with the same reference numerals. The present disclosure relates to nasojejunal (NJ) tubes and/or catheter devices including features for retention at a target location in the gastrointestinal (GI) tract.

Pyloric stenosis involves a narrowing of the gastric outlet (pylorus) which may cause difficulties in passing food from the stomach to the small bowel. This may lead to gastric outlet obstruction (stricture) reducing or completely impeding the ability of the patient to digest food and absorb nutrients. Similar obstructions may also occur in the small bowel, e.g., the duodenum.

As indicated above, there are several currently employed treatments for this problem, including placing a nasojejunal (NJ) feeding tube in the small bowel. The NJ tube is fed down the esophagus into the stomach, e.g., via endoscope or guidewire, and guided through the stricture so that nutrients may be provided to the small bowel, e.g., the jejunum, using the tube to bypass the stricture. The NJ tube typically comprises holes along a length of a portion of the tube near the distal end of the tube so that this portion, when positioned as desired, discharges nutrients from the tube into the target anatomical structure (e.g., small bowel) via the holes.

FIG. 1a shows a nasojejunal (NJ) tube device 100 according to one example. The device includes a catheter 102 (e.g., tube) extending from a proximal end (not shown) to a distal end 104 with, for example, an atraumatic distal tip, the catheter 102 being configured to be advanced through the gastrointestinal (GI) tract (e.g., sized and shaped to be inserted from a natural body orifice through a body lumen until a distal end of the catheter reaches a target site within the body while proximal end of the catheter remains outside the body accessible to a user). The catheter 102 of this example comprises a single lumen or channel although those skilled in the art will understand that additional lumens may be included along all or any part of the length of the catheter 102 if desired.

The catheter 102 has a distal portion 106 extending proximally from the distal end 104 for a distance selected to include one or more holes 108 configured to discharge a fluid administered through the lumen of the catheter 102 into a target anatomical structure. In short, the distal portion 106 of the catheter 102 extends along a portion of the catheter 102 that is to be positioned within a target anatomical area into which fluids are to be supplied via the catheter 102. The catheter 102 of this embodiment is configured to be guided through the GI tract (e.g., through the mouth and along the esophagus through the stomach into the small bowel) to a position in which the distal portion 106 is located in the small bowel, e.g., the jejunum.

The NJ tube device 100 further comprises a handle 110 including a port 112 in fluid communication with and configured to access the lumen of the catheter 102 (i.e., to remain accessible to a user when the distal portion of the catheter 102 is at the target location), e.g., to administer the fluid from the port 112 to the target anatomical structure via the lumen of the catheter 102. The catheter 102 is configured (sized and shaped) to be advanced longitudinally through a channel of the handle 110 as the catheter 102 is guided to the target location. The proximal end of the catheter 102 is fixed to a cap 114 configured to interface with the handle 110 when the catheter 102 is fully extended distally relative to the handle 110.

FIG. 1b shows patient anatomy 120 prior to inserting a NJ tube according to one example. The anatomy 120 comprises a portion of the GI tract including the esophagus 122, the stomach 126 and the small bowel 130 (small intestine). The small bowel 130 includes the duodenum 132, the jejunum 136, and the ileum (not shown). As would be understood by those skilled in the art, the esophageal sphincter 124 corresponds to the end of the esophagus 122 and the start of the stomach 126, the pylorus 128 corresponds to the end of the stomach 126 and the start of the duodenum 132 and the ligament of Treitz 134 is a ligament supporting the small bowel and corresponds to the end of the duodenum 132 and the start of the jejunum 136. Pyloric stenosis refers to a narrowing of the pylorus 128 that can create a stricture 138 preventing or making difficult the passage of food from the stomach 126 to the small bowel 130.

FIG. 1c shows the catheter 102 of the NJ tube device 100 of FIG. 1a placed in the anatomy 120 of FIG. 1b according to one example. The catheter 102 of the NJ tube device 100 is configured to be passed down the esophagus 122, through the stomach 126, past the stricture 138 (e.g., at the pylorus 128 which has narrowed due to any of a variety of causes) and into the small bowel 130 under visualization (e.g., via endoscope) by using a guidewire, and/or by other methods. The catheter 102 is configured to be passed through the duodenum 132 past the ligament of Treitz 134 to the jejunum 136 so that the distal portion 106 comprising the holes 108 is positioned at least partially in the jejunum 136. In other cases, the catheter 102 can be placed further proximally in the duodenum. Once the catheter 102 has been placed at the desired location, fluids can be administered via the handle 110 and discharged into the jejunum 136 via the holes 108.

In this example, the NJ tube device 100 is for feeding purposes and the fluids administered by the tube comprise nutrients for the patient. In other examples, the fluid can comprise contrast or other therapeutic fluids, e.g., medicines, fluids configured to enhance visualization for assisting endoscopic ultrasound guided gastroenterostomy (EUS-GE) procedures, etc.

Another method for treating a patient with pyloric stenosis includes the creation of a surgical gastro-jejunostomy bypass bridging the stomach and the jejunum to bypass the area of the stricture by permitting material to move directly from the stomach into the small bowel downstream of the stricture. NJ tubes can be used to assist with EUS-GE procedures by instilling contrast or another fluid into the jejunum to enhance visualization of the anatomy. In one example, contrast is provided to enhance visualization of a target by an operating physician, e.g., for a procedure including piercing the stomach and the jejunum and placing a stent (e.g., an AXIOS stent) therebetween to bypass the pylorus. It is noted that EUS-GE procedures may alternatively target the duodenum and any reference to the jejunum as the target location is for exemplary purposes only.

FIG. 2 shows an EUS-GE system 150 for placing a gastro-jejunal stent 162 in the anatomy 120 of FIG. 1b according to one example. The EUS-GE system 150 comprises a NJ tube device 152 including a catheter 154 that is passed into the jejunum 136 as described above so that fluid can be discharged into the jejunum 136. In this example, the fluid comprises contrast fluid 156 to help the operating physician visualize the target location as would be understood by those skilled in the art.

The EUS-GE system 150 of this example further comprises an endoscope 160 for deploying the stent 162. As would be understood by those skilled in the art, a piercing element (not shown) can be deployed via the endoscope 160 to pierce the stomach 126 and the jejunum 136. The stent 162 is placed through the openings made by the piercing element with a proximal end of the stent 162 remaining in the stomach 126 while the distal end of the stent 162 is anchored in the jejunum 136 providing a direct path for material transport from the stomach 126 to the jejunum 136 bypassing the pylorus 128.

One issue with NJ tubes is such tubes may drift out of position during use such that fluids discharged by the tube are provided to unintended locations. In one example, the NJ tube migrates proximally during use. In feeding tube applications, this proximal drift can cause nutrients to be provided further up in the small bowel, or even in the stomach proximal to the stricture, which would cause issues with digestion.

In EUS-GE applications, the drift of the NJ tube can cause fluids (e.g., contrast fluid) to be instilled away from an intended target area. With regard to the gastro-jejunostomy procedure described in FIG. 2, this prevents the contrast fluid from assisting in the proper visualization of the target anatomy. This may lead to difficulties in the procedure such as the mis-deployment of a stent, etc. In some examples, the proximal end of the stent may be positioned properly in a hole created in the stomach while the distal end of the stent fails to anchor in the jejunum (which may or may not have been pierced by the piercing element); similarly, the distal end of the stent may be properly anchored in the jejunum while the proximal end of the stent fails to anchor as desired within the stomach; or the stent may be deployed to bridge the stomach to a non-targeted organ such as the colon. Accordingly, there is a need for improved devices and mechanisms to retain a tube at a desired location in the GI tract.

According to various exemplary embodiments, systems, devices and methods are described for retaining a catheter, e.g., a nasojejunal tube, at a desired location in the small bowel. The catheter can be retained at a position in the small bowel such that the holes for discharging a fluid are positioned in a desired location, e.g., the jejunum. Some of the devices described herein include a catheter intended for use as a feeding tube, e.g., for extended use (weeks, months, etc.), and some of the devices described herein include catheters intended for the provision of fluid during EUS-GE procedures, as will be described below, where the catheter is removed after the procedure. However, it is noted that many of the described embodiments are equally applicable for either purpose, as described in detail below.

The terms “NJ tube” and/or “catheter” are used herein to describe a tube configured for deployment within the GI tract for administering a fluid, e.g., for feeding, a therapeutic fluid or a fluid applied to facilitate a procedure such as EUS-GE. The catheters according to the present embodiments generally have a single lumen or channel for passing a fluid through the catheter from a proximal end to a distal portion from which the fluid can be discharged through holes in the wall of the catheter. In some cases, the catheter may not have holes in its wall but rather may have an open distal tip for discharging the fluid. Alternatively, a catheter may have both a series of holes located along a length of a distal portion thereof and an open distal end.

Those skilled in the art understand that a simple catheter can comprise a length of tube, e.g., formed of a material such as a plastic and/or polymer (e.g., PVC, PTFE, etc.) with a desired flexibility and/or rigidity, that may assume a substantially straight (longitudinal) or slightly curved shape when no external forces are applied at any location along the length of the tube. However, the simple catheter is typically sufficiently flexible such that during deployment the catheter can bend or curve under external forces imposed by, e.g., an operating physician at the proximal end of the catheter; by contacting an inner wall of an endoscope channel; by contacting inner walls of organs along the GI tract; or by passing the catheter over a guidewire.

Accordingly, the simple catheter can assume many variations of three-dimensional shapes due to its flexibility and various external forces imposed at various locations along its length allowing the catheter to extend along a tortuous path. However, those skilled in the art understand that the ability of the catheter to curve or bend is generally limited and such bending typically proceeds in a snake-like manner, e.g., as body lumens are traversed. Those skilled in the art will ascertain that a simple catheter generally does not expand in shape, for example, radially, nor does a typical simple catheter have additional features for expansion relative to its circular cross-section along its length.

In various exemplary embodiments described herein, a catheter device includes a material attached to or deployable from the catheter which material is designed to anchor the catheter at a desired location in the GI tract. In some embodiments, the material can be transitioned from a first state or shape suitable for advancing the catheter through the GI tract to the target location into a second state or shape suitable for engaging the inner wall of the target location.

In one aspect of these exemplary embodiments, a catheter device includes a catheter with an outlet for a fibrous gel or glue that can be injected into the small bowel whereupon the gel/glue forms a fibrous bundle configured to engage an inner wall of the small bowel. In one embodiment, the gel/glue is selected so that the gel/glue, when activated, solidifies into the fibrous bundle upon injection into the small bowel, e.g., activation by contact with materials (e.g., fluid or gas) present in the small bowel. In another embodiment, the glue comprises components that are held separately until mixed upon deployment to form the gel/glue on site, e.g., at or adjacent to the outlet of the catheter. The amount of material is selected to form a fibrous bundle of a size sufficient to engage and temporarily stick to the catheter and the inner wall of, e.g., the jejunum, to anchor the catheter in place.

Those skilled in the art will ascertain that the fibrous gel/glue be selected from any of a number of different materials including, e.g., hydrogels or fibrin glues known in the art. These materials are biocompatible so that they do not evoke an immune response and can be passed through the GI tract when no longer used for the anchoring functionality. The fibrous bundle can break down over time or, in some embodiments, can be broken down by introducing a solvent through the catheter.

FIG. 3 shows a catheter device 200 including a fibrous bundle 214 formed from a fibrous gel or glue discharged from a catheter 202 within the patient anatomy of FIG. 1b according to a first example of these exemplary embodiments. The catheter device 200 is configured to be introduced into the esophagus via the mouth, passed through the stomach past the stricture and placed such that a distal end 204 of the catheter 202 is within the small bowel. The anatomy 120 shown in FIG. 3 includes the jejunum 136 which is the target anatomical location in this example.

The catheter 202 extends from a proximal end (not shown) to a distal end 204 with an atraumatic distal tip. A first distal portion 206 of the catheter 202 includes multiple holes (not labeled) providing an outlet for fluid introduced via a first channel 208 (first lumen) of the catheter 202. In other examples, the first distal portion 206 comprises a single hole. A second distal portion 210 of the catheter 202 includes multiple holes (not labeled) providing an outlet for fluid or gel introduced via a second channel 212 (second lumen). In this example, the second distal portion 210 is distal to the first distal portion 206.

When the second distal portion 210 is positioned at a target location in the jejunum 136 the fibrous gel/glue can be instilled via the second channel 212. In one example, a gel is injected into the jejunum 136 where it is activated by the gases/fluids therein. In another example, individual components for a glue are mixed into the glue at or adjacent to the outlet of the second distal portion 210. In one example, the individual components could be injected from two different lumens until reaching the distal tip, where they are then mixed. In another example, the gel/glue may have a delayed reaction so that it does not activate until after injection. In some examples, the gel/glue may behave like a two-part epoxy.

The gel/glue solidifies into the fibrous bundle 214 comprising an enlarged volume sized to engage the inner wall of the jejunum 136 and anchor the catheter 202 in place. The fibrous bundle 214 may also be sticky to better adhere to the catheter 202 and the inner wall of the jejunum. After anchoring the catheter 202 in place, fluid can be provided via the first channel 208 and discharged via the holes in the first distal portion 206 of the catheter 202. The fibrous bundle 214 also helps prevent the fluid from passing distally beyond the distal end of the catheter 202 so that fluid is retained in a target location (e.g., a portion of the jejunum proximal to a distal end of the catheter 202.

In this example, the desired location for discharging the fluid is within the jejunum 136, e.g., for assisting an EUS-GE procedure such as that discussed with regard to FIG. 2. The fibrous bundle 214 is deployed from the second distal portion 210 to provide an anchor location distal to the desired location. Those skilled in the art will understand that, in other embodiments, the second distal portion 210 and fibrous bundle 214 may be located differently on the catheter 202 relative to the first distal portion 206 comprising the fluid outlet. For example, the second distal portion 210 may be located proximally to the first distal portion 206 such that the anchor location is proximal to the hole(s) for discharging the fluid, or the first distal portion 206 and second distal portion 210 can coincide wholly or in part.

In this example, the fluid comprises a contrast fluid or saline to assist in visualizing the small bowel 130. However, it should be understood that a similar catheter device may be deployed as a feeding tube so that the first distal portion 206 and/or the second distal portion 210 is placed further proximally, e.g., in the duodenum or toward the beginning of the jejunum, so that a fluid comprising nutrients can be provided at a location where the nutrients will be digested.

In this example, the catheter 202 has a closed distal end 204 such that only two channels/lumens are used. In other embodiments, the catheter can include a third channel comprising a guidewire lumen.

In another aspect of these exemplary embodiments, a catheter device includes a time-activated adhesive on an outer surface thereof. In one embodiment, this adhesive is applied to the tube as a thin layer so that it does not create an obstruction (e.g., does not substantially increase the cross-sectional area of the catheter) that might hinder the placement of the catheter in a desired location. In another embodiment, the adhesive is applied within one or more indentations along the catheter which also avoids any obstruction from the adhesive during placement while allowing the adhesive to interact with and react to the materials in the body lumens to become sticky.

The adhesive may be composed of materials with a selected intubation time (activation time required before the adhesive assumes its adhesive qualities) based on the crosslinking of the adhesive components. In one example, a material composition comprising chitosan with polyethylene glycol is formulated to have an activation time anywhere between 2 minutes and two hours as would be understood by those skilled in the art.

FIG. 4a shows a catheter device 220 including a catheter 222 with a second distal portion 226 on which an adhesive 228 is applied according to a second example of these exemplary embodiments; FIG. 4b shows the adhesive 228 of the catheter device 220 of FIG. 4a according to the second exemplary embodiment; FIG. 4c shows a second distal portion 232 of a catheter device 230 including indentations 234 configured to hold adhesive at positions distributed along the second distal portion 232 according to a third example of these exemplary embodiments. Similar to the catheter devices of the preceding embodiments, the catheter devices 220, 230 are configured to be introduced into the stomach via the esophagus and passed through the stricture so that a distal end of the catheter extends within the small bowel.

The catheter 222 extends from a proximal end (not labeled) at a handle 229 of the catheter device 220 to a distal end 224 with an atraumatic distal tip, as shown in FIG. 4a. A first distal portion (not labeled) of the catheter 222 provides an outlet for fluid introduced via the lumen of the catheter 222 that comprises one or more holes for discharging the fluid to the target location. A second distal portion 226 includes an adhesive 228 (e.g., a time activated adhesive) applied to its exterior, as shown in FIG. 4b. It is noted that the first distal portion is, in this embodiment, located proximally to the second distal portion 226 although, as would be understood by those skilled in the art, the first distal portion may alternatively be located distally to the second distal portion 226, or the first and second distal portions may be located overlap of coincide in whole or in part along the length of the catheter device 220.

The catheter 222 may be advanced through the GI tract within the intubation time of the adhesive 228 so that the adhesive 228 has not yet assumed its adhesive qualities while traversing the GI tract until the adhesive 228 is positioned adjacent to the target location. After the intubation time, the adhesive 228 becomes sticky and the second distal portion 226 of the catheter 222 can then engage the inner wall of the jejunum to anchor the catheter 222 in place. After the catheter 222 has been anchored in place, fluid may be provided via the lumen and discharged via the holes in the first distal portion of the catheter 222 to the target location.

Similar to the catheter devices of the preceding embodiments, the catheter device 220 is configured for use in providing fluid for enhancing visualization of the the small bowel for an EUS-GE procedure or can be placed further proximally for use as a feeding tube. In the EUS-GE applications, the catheter device 220 can provide the physician a target that is visible under ultrasound. In some embodiments, the catheter can comprise markings that are visible under direct imaging and/or fluoroscopy to help identify where the distal tip of the device is located. In other embodiments, the catheter can comprise markings near the handle for the same purpose.

In another aspect of these exemplary embodiments, a catheter device includes a superabsorbent material attached thereto such that, as fluid is absorbed, the material expands to engage the tissue of the target anatomical structure to anchor the catheter at the desired location. In one example, the superabsorbent material comprises a superabsorbent polymer cross linked with an acrylic homopolymer to enable the material to stretch and expand as water is absorbed. When the distal tip of the catheter is in position past the obstruction it will be held in position for an acute period of time.

Over this period of time, the superabsorbent polymer will absorb fluid within the gut. Additional water could be fed into the GI tract to allow for the distal tip to absorb fluid more quickly. As it absorbs the fluid the material will expand, increasing the outer diameter of the distal end of the catheter. The amount and configuration of this material is selected so that, as the material expands, the resultant expanded size of the material is sufficient to prevent the distal portion of the catheter from being pulled back through the stricture but not so much that is obstructs the lumen. Thus, as the patient drinks water or water is fed through the NJ tube the distal tip of the catheter will be kept sufficiently engorged with water, to ensure that the distal portion of the catheter remains distal of the stricture. As would be understood by those skilled in the art, when the catheter is ready to be removed, the patient may cease drinking water for a selected period of time until the material dries and shrinks so that the catheter can be removed. If the stricture has been removed or reduced the catheter may simply be retracted proximally without shrinking the superabsorbent material.

FIG. 5a shows a catheter device 240 including a superabsorbent expanding material 249 attached to a catheter 242 in a contracted state (first shape) within the patient anatomy of FIG. 1b according to a fourth example of these exemplary embodiments; FIG. 5b shows the material 249 of the catheter device 240 in an expanded state (second shape) within the patient anatomy of FIG. 1b according to the fourth exemplary embodiment. The catheter device 240 can be introduced in the esophagus and passed through the stomach past the stricture 138 and placed such that a distal end 244 of the catheter 242 is within the small bowel 130. In this example, the distal end 244 of the catheter 242 may be placed further proximally, or may be allowed to drift further proximally, relative to the preceding embodiments, as will be explained in detail below.

The catheter 242 extends from a proximal end (not shown) to a distal end 244 with an atraumatic distal tip. A first distal portion 246 of the catheter 242 includes multiple holes (not labeled) for providing an outlet for fluid introduced via a lumen of the catheter 242. In other examples, the first distal portion 246 may comprise a single hole for providing an outlet for fluid introduced via a lumen of the catheter 242. A second distal portion 248 of the catheter 242 includes a surface over which the material 249 can be attached. In this example, the second distal portion 248 coincides in part with the first distal portion 246.

The catheter 242 can be advanced through the GI tract with the material 249 contracted, e.g., prior to the material 249 absorbing a sufficient amount of fluid to expand, until the material 249 is positioned at a target location. Water can be introduced to the GI tract so that the material 249 can absorb the water and expand. After a sufficient amount of fluid has been absorbed, the material 249 will expand to a sufficient degree to prevent passing proximally through the stricture 138. Yet the amount and configuration of the material 249 is selected so that it will not expand to such a degree that the material 249 anchors the catheter 242 in place or blocks the lumen of the target organ. Fluid can be provided via the lumen and discharged via the holes in the first distal portion 246 of the catheter 242.

The catheter device 240 can be used as a feeding tube. So long as the nutrients are delivered distal to the structure they can be digested properly.

In another aspect of these exemplary embodiments, a catheter device includes a sponge attached around suction ports on a catheter used to distribute suction force in the lumen of the duodenum to stabilize and prevent migration of the catheter during placement of a gastro-jejunal stent (e.g., an AXIOS stent). Attracting the duodenal wall towards the catheter using a sponge, rather than pushing outwards on the duodenal wall using a radial force, helps create traction without interfering with positioning of the anatomy, making it easier for physicians to accurately target and achieve gastro-jejunal stent placement. In some embodiments, the catheter device includes a retractable sheath for uncovering the sponge. In some embodiments, the catheter device includes a balloon distal to the sponge such that the balloon can occlude the small bowel at a distal location (e.g., in the jejunum) and the sponge can occlude the small bowel at a proximal location (e.g., in the duodenum) to prevent proximal and/or distal migration of the catheter.

FIG. 6a shows a catheter device 250 including a sponge 276 attached to a catheter 260 for distributing a suction force in a fully contracted state (first shape) according to a fifth example of these exemplary embodiments; FIG. 6b shows the catheter device 250 of FIG. 6a with the sponge 276 in an expanded state (second shape) according to the fifth exemplary embodiment; FIG. 6c shows the catheter device 250 of FIG. 6a with the sponge 276 in a partially contracted state (third shape) under the suction force within the patient anatomy of FIG. 1b according to the fifth exemplary embodiment; FIG. 6d shows the sponge 276 of the catheter device 250 of FIG. 6a in the partially contracted state (third shape) within the patient anatomy of FIG. 1b according to the fifth exemplary embodiment.

The catheter 260 extends from a proximal end 262 to a distal end 264 with an atraumatic distal tip that, in this example, is open to a guidewire lumen (not labeled) of the catheter 260 such that the catheter 260 can be passed over a guidewire to the target location. In other embodiments, the distal end 264 of the catheter 260 can be closed and the catheter device 250 can be advanced to the target location under alternative visualization methods that do not employ a guidewire as would be understood by those skilled in the art.

In this example, the catheter 260 includes a first distal portion 266 comprising suction ports 267 along its length open to a first channel (not shown) for applying a suction force via a first proximal port 268. The catheter device 250 further includes the sponge 276 disposed around the suction ports 267 so that the suction force applied to the suction ports 267 will be distributed throughout the sponge 276, to be described in greater detail below. The catheter 260 further includes a second distal portion 270 including multiple holes (not labeled) for providing an outlet for fluid introduced via a second proximal port 272 through a second channel (not shown) of the catheter 260.

In this example, the catheter device 250 further includes a balloon 278 disposed around a third distal portion 274 of the catheter 260 adjacent to the distal end 264. A proximal end (not labeled) of the balloon 278 is open to a third channel (not labeled) for inflating the balloon 278 with fluid (e.g., saline) via a third proximal port (not labeled) at the proximal end 262 of the catheter 260. In this example, the third distal portion 274 comprising the balloon 278 is distal to the second distal portion 270 comprising the exit ports, the second distal portion 270 being distal to the first distal portion 266 comprising the suction ports 267 and the sponge 276. The catheter device 250 further includes a sheath 252 for covering/uncovering the sponge 276. The sheath 252 extends from a proximal end 254 to a distal end 256 and includes a fourth channel 258 through which the catheter 260 can be received and translate proximally/distally.

As shown in FIG. 6a, when the sheath 252 is in a first position (distal position) the sheath 252 constrains the sponge 276 around the first distal portion 266 of the catheter 260 in a first state or shape that is fully contracted. The catheter device 250 can be introduced into the GI tract with the sponge 276 encased within the sheath 252. The balloon 278 can be fixed to the third distal portion 274 of the catheter 260 at its proximal and distal ends. Prior to inflation, the balloon 278 can remain in a deflated state (first shape) suitable for advancing the distal end 264 of the catheter 260 past the stricture 138 and into the small bowel such that the deflated balloon 278 is in a distal position in the small bowel and the sponge 276 is in a proximal position in the small bowel, to be described in greater detail below. In this example, to transition the balloon 278 into an inflated state (second shape), fluid is provided via the third channel to fill the inner volume of the balloon 278.

As shown in FIG. 6b, the sheath 252 can be retracted proximally to expose the sponge 276 and remove the constraint of the sheath 252 such that the sponge 276 expands radially from the first distal portion 266 of the catheter 260 to engage the inner walls of the small bowel at a first (proximal) location, e.g., the duodenum. The balloon 278 can be inflated such that the balloon 278 expands radially from the third distal portion 274 of the catheter 260 to engage the inner walls of the small bowel at a second (distal) location, e.g., the jejunum.

As shown in FIG. 6c, the catheter device 250 is configured for introduction into the small bowel 130. The balloon 278 is expanded to occlude the small bowel 130 at the distal location while suction is applied under the sponge 276 to occlude the small bowel 130 at the proximal location to prevent proximal/distal migration of the catheter 260. As shown in FIG. 6d, the suction force draws the inner walls of the small bowel 130 radially inward toward the catheter device 250. Once in place, contrast or other fluid can be injected through the exit ports of the second distal portion 270, e.g., to fill the space within the small bowel extending between the sponge 276 and the balloon 278.

Similar to the catheter devices of the preceding embodiments, the catheter device 250 is configured to provide fluid for enhancing visualization of the small bowel for an EUS-GE procedure. At a suitable time during or after completion of the EUS-GE procedure, the balloon 278 can then be deflated and the sponge 276 can be re-sheathed so that the catheter device 250 may be withdrawn from the GI tract. It is noted that the sponge could be thoroughly dried during manufacturing to shrink it down and may not be exposed to fluid until deployment. The catheter may have a funnel tip shape to help enable recapture of the sponge. Alternatively, the sponge could be pulled out of the body without recapturing.

In another aspect of these exemplary embodiments, the sponge can be made from or incorporate an auxetic material to ease delivery. One or more sections of auxetic material could be incorporated into the catheter near where the sponge would need to deploy. Either pushing or pulling on the sponge would cause the sponge to expand radially due to its material structure. This nonconventional behavior would allow the sponge to be delivered in a low-profile way while allowing the sponge to also contribute radial expansive force (like a balloon) in addition to suction dispersion. Given this behavior, an alternative embodiment could incorporate two sponges to anchor the catheter in place, rather than a proximal sponge and a distal balloon.

FIG. 7a shows a conventional material 280 according to one example. The conventional material 280 in the shape of a cylinder compresses radially as it elongates under a pulling force.

FIG. 7b shows an auxetic material. 282 according to a seventh example of these exemplary embodiments. The auxetic material 282 in the shape of a cylinder expands radially as it elongates under a pulling force.

In still another aspect of these exemplary embodiments, a catheter device includes a clip, e.g., a resolution clip, deployable from a distal end of the catheter via a coil. The clip is maneuvered by the coil, which is coupled to the handle and can be manipulated by the user to provide rotation and can elongate to aid in the deployment of the clip. The catheter can be placed at a target location and the clip can be fed through the lumen of the catheter to a hole at the distal end of the catheter. The clip can be rotated by rotating the handle, coil or both. The clip includes jaws that can be opened and closed by the handle at the proximal end of the coil. The catheter device can include a wire or tether that couples the distal tip of the catheter to the clip. The clip can be deployed to engage the inner wall of the small bowel. Once the clip is deployed the coil is removed and the clip tethered to the catheter remains, ensuring the distal tip of the catheter does not migrate from its target location.

FIG. 8a shows a catheter device 290 including a clip 298 deployable from a distal end 294 of a catheter 292 in a withdrawn state within the distal end 294 within the anatomy 120 of FIG. 1b according to an eighth example of these exemplary embodiments; FIG. 8b shows the catheter device 290 of FIG. 8a with the clip 298 in a partially deployed state within the anatomy 120 of FIG. 1b according to the eighth exemplary embodiment; FIG. 8c shows the catheter device 290 of FIG. 8a with the clip 298 in a fully deployed state engaging the inner wall of the small bowel 130 within the anatomy 120 of FIG. 1b according to the eighth exemplary embodiment. The catheter device 290 can be introduced in the esophagus and passed through the stomach past the stricture 138 and placed such that a distal end 294 of the catheter 292 is within the small bowel 130.

The catheter 292 extends from a proximal end (not shown) to a distal end 294 with an atraumatic distal tip. The clip 298 can be housed within a cavity in the distal end 294, as shown in FIG. 9a. A distal portion 296 of the catheter 292 includes multiple holes (not labeled) for providing an outlet for fluid introduced via a lumen of the catheter 292. In other examples, the distal portion 296 can comprise a single hole.

The catheter 292 can be advanced through the GI tract with the clip 298 contained within the distal end 294 of the catheter. When the distal end 294 is positioned at the target location, the clip 298 can be deployed, as shown in FIGS. 8b-c. A capsule comprises the clip 298 and a yoke that releases when the clip 298 is pulled back by the coil 300. The clip 298 is ejected from the distal tip when the handle spool is pulled proximally, and the yoke is overcome. The coil 300 is released when the clip 298 is deployed. As would be understood by those skilled in the art, the clip 298 comprises jaws 302 that open when the jaws 302 are not constrained by the catheter 292 as the clip 298 is moved distally out of the catheter 292. The jaws 302 are closed when the clip 298 has traveled a given distance out the distal end 294. The clip 298 can engage the inner wall of the small bowel 130 when the jaws 302 close to anchor the catheter 292 in place. Fluid can be provided via the lumen and discharged via the holes in the distal portion 296 of the catheter 292.

Similar to the catheter devices of the preceding embodiments, the catheter device 290 can be used for providing fluid for visualizing the small bowel for an EUS-GE procedure or can be placed further proximally for use as a feeding tube.

In still another aspect of these exemplary embodiments, a catheter device includes a semi-rigid disk which is slit through in the center region. The catheter is passed through this slit/opening, which then applies a degree of compression to the catheter, e.g., due to an interference fit. The disk is positioned on the catheter at a location on the catheter that is separated proximally from the distal end of the catheter by a length corresponding to a distance along the catheter from the target location of the distal end of the catheter to a distal side of the esophageal sphincter. The catheter is placed normally, and carries the disk along with it, due to the interference fit with the tube. The material of the disk has been selected so that, when the disk reaches the esophagus, the flexibility of the disk allows it to fold and bend such that it can pass through the esophagus.

Once the disk is distal to the esophageal sphincter, it can unfold and expand to its full & original shape. The position of the disk then provides a degree of interference to prevent the catheter from migrating in reverse, as the disk provides a physical barrier that interferes with the esophageal sphincter to prevent inadvertent movement of the catheter proximally beyond this position. Thus, due to the longitudinal position of the disk on the catheter, when the catheter is pulled proximally until the disk engages the esophageal sphincter, the distal end of the catheter is located at a target location in the target organ and the catheter is prevented from moving proximally beyond this location.

FIG. 9a shows a catheter device 310 including a disk 314 disposed around a catheter 312 at a location selected so that, when the distal portion of the catheter device 310 is in a target location within the anatomy 120 of FIG. 1b the disk is distal of and adjacent to the esophageal sphincter 124 according to a ninth example of these exemplary embodiments; FIG. 9b shows the disk 314 of the catheter device 310 of FIG. 9a according to the ninth exemplary embodiment. The catheter device 310 can be introduced in the esophagus and passed through the stomach past the stricture and placed such that a distal end (not labeled) of the catheter device 310 is within the small bowel 130 in the same manner described above.

The catheter 312 extends from a proximal end (not shown) to a distal end (not labeled) with an atraumatic distal tip. A distal portion (not labeled) of the catheter 312 can include one or more holes for providing an outlet for fluid introduced via a lumen of the catheter 312. The disk 314 comprises a slit 316 that can receive the catheter 312 and attach thereto with an interference fit. The disk 314 is located on the catheter 312 so that the disk 314 will advance past the esophageal sphincter 124 when the distal end is advanced to the target location. The size and flexibility of the disk 314 is selected such that the disk 314 deforms to permit the disk 314 to pass distally beyond the esophageal sphincter 124 yet provides resistance to the catheter 312 sufficient to withstand the internal bodily forces applied to the catheter 312 that might otherwise result in proximal migration of the catheter 312 (i.e., such anticipated forces are insufficient to push the disk 314 proximally past the esophageal sphincter 124).

Similar to the catheter devices of the preceding embodiments, the catheter device 310 can be used for providing fluid for visualizing the small bowel for an EUS-GE procedure or can be placed further proximally for use as a feeding tube.

In still another aspect of these exemplary embodiments, an endoscopic device includes an accessory device comprising semi-rigid material in the form of a C-channel which mates with an exterior of an endoscope and can carry a catheter, e.g., a nasogastric tube. The C-channel includes at least one (or perhaps multiple) lumens through which catheters such as nasogastric tubes may be placed. In this embodiment, the C-channel may be attached to the endoscope prior to introduction to the patient. The C-channel may be secured to the outside of the scope via an interference fit and/or via securement material such as tape that encircles both the C-channel and endoscope at multiple points along the length of both devices.

The catheter can then be introduced through the lumen of the C-channel, such the that distal end of the catheter is just distal to the end of the endoscope. The scope, C-channel, and catheter can then be introduced into the patient and advanced to the desired location together. If the catheter is desired to travel beyond the end of the scope, then it may be advanced (over a wire or not) further distal, continually passing through the C-channel until it reaches its desired location. For removal, the catheter may be pulled proximally with the C-channel remaining stationary. Alternatively, the C-channel, scope, and catheter may be pulled proximally as a complete unit for device removal.

FIG. 10a shows an accessory device 320 for an endoscope 170 comprising a channel 322 through which a catheter can be passed according to a tenth example of these exemplary embodiments; FIG. 10b shows a cross section of the accessory device 320 of FIG. 10a according to the tenth exemplary embodiment.

The accessory device 320 can be attached to an endoscope 170 by the surface 324 and introduced into the patient anatomy. The surface 324 provides, for example, an interference fit with the endoscope 170. In one example, a catheter is passed through the channel 322, the catheter comprising a nasogastric or nasojejunal feeding tube for instilling fluid in the small bowel, e.g., saline/contrast or nutrients. In other embodiments, the accessory device 320 comprises multiple channels for multiple catheters and/or other devices.

It may be noted by those knowledgeable in the art that any of the above embodiments may be combined in any manner not inconsistent with their operation and design to provide a system to enable ostomy management utilizing any or all of the characteristics of the various embodiments.

1-15. (canceled)

16. A device for administering a fluid to a patient anatomy, comprising:
a flexible catheter including at least a first channel extending therethrough, the catheter being sized and shaped to extend from a proximal end outside a human body to a distal end that can be guided to a target location within a gastrointestinal (GI) system, a first distal portion of the catheter including one or more exit ports communicating with the first channel for administering a fluid; and
a material fixed to an exterior of the catheter or ejectable from an interior of the catheter into the target location that, in a first state or shape, is suitable for guiding the catheter to the target location and, in a second state or shape, is suitable for engaging an inner wall of the target location to anchor the catheter in the target location while administering the fluid via the first channel.

17. The device of claim 16, wherein the catheter includes a second distal portion including one or more exit ports communicating with a second channel, the second distal portion distal to the first distal portion, the material comprising a fibrous gel or a fibrous glue injected through the second channel,
wherein, in the first state or shape, the fibrous gel or the fibrous glue behaves as a gel or fluid for traversing the second channel, and
wherein the fibrous gel or the fibrous glue is discharged out of the exit ports of the second distal portion and transitions into the second state or shape in which the fibrous gel or the fibrous glue solidifies into a fibrous bundle for engaging the inner wall of the target location.

18. The device of claim 17, wherein the fibrous gel or the fibrous glue is activated to transition into the fibrous bundle by fluid or gas in the GI system after discharge into the target location.

19. The device of claim 17, wherein the fibrous glue includes components that are mixed upon discharge into a target volume to transition into the fibrous bundle.

20. The device of claim 16, wherein the catheter includes a second distal portion at which the material is applied, the material comprising an adhesive, wherein, in the first state or shape, the adhesive behaves as a solid without adhesive qualities, and wherein the adhesive transitions into the second state or shape and behaves with the adhesive qualities after a duration suitable for guiding the catheter to the target location.

21. The device of claim 20, wherein the adhesive comprises a time-activated material with a selected intubation time for transitioning into the second state.

22. The device of claim 21, wherein the second distal portion of the catheter includes longitudinal indentations for applying the adhesive.

23. The device of claim 16, wherein the catheter includes a second distal portion at which the material is attached, the material comprising a flexible super-absorbent material, wherein, in the first state or shape, the material is contracted around the second distal portion, and
wherein the material absorbs a further fluid to transition into the second state or shape in which the material expands.

24. The device of claim 23, wherein, in the second state or shape, the material is of a size sufficient to prevent the material from passing proximally through a stricture in the GI system.

25. The device of claim 16, wherein the catheter includes a second distal portion at which the material is attached and further includes suction ports, the catheter including a second channel extending therethrough communicating with the suction ports, the material comprising a sponge,
wherein, in the first state or shape, the sponge is fully contracted around the second distal portion,
wherein, in the second state or shape, the sponge is released from the first state or shape and expands to engage the inner wall of the target location, and
wherein, in a third state or shape, a suction force is applied via the suction ports to distribute the suction force throughout the sponge to partially contract the sponge and the inner wall of the target location.

26. The device of claim 25, further comprising:
a sheath disposed around the second distal portion of the catheter, the sheath being translatable relative to the catheter from a first position to a second position, wherein the sponge is constrained by the sheath around the catheter in the first state or shape when the sheath is in the first position and translating the sheath into the second position unconstrains the sponge to allow the sponge to assume the second state.

27. The device of claim 25, further comprising:
a balloon adjacent to the distal end of the catheter, the catheter including a third channel extending therethrough communicating with an interior of the balloon so that, in a first state, the balloon is deflated and assumes a first shape suitable for guiding the distal end of the catheter to a further target location distal to the target location and, in a second state, the balloon is inflated and assumes a second shape in which the balloon extends radially from the catheter for contacting an inner wall of the further target location to anchor the catheter at the further target location,
wherein the first distal portion including the exit ports is distal to the sponge and proximal to the balloon.

28. The device of claim 25, wherein the sponge is formed from or incorporates an auxetic material that expands radially under tension or compression to transition the sponge into the second state or shape.

29. The device of claim 16, further comprising:
a clip housed within a cavity at the distal end in the first state, the clip being deployable from the distal end so that, in the second state, the clip grasps the inner wall of the target location.

30. The device of claim 29, wherein the clip is deployable by a coil and remains fixed to the catheter via a tether.

31. A method for administering a fluid to a patient anatomy, comprising:
guiding a flexible catheter to a target location within a gastrointestinal (GI) system, the catheter including at least a first channel extending therethrough, the catheter being sized and shaped to extend from a proximal end outside a human body to a distal end, a first distal portion of the catheter including one or more exit ports communicating with the first channel for administering a fluid;
transitioning a material from a first state or shape to a second state or shape, the material being fixed to an exterior of the catheter or ejectable from an interior of the catheter into the target location, the first state or shape being suitable for guiding the catheter to the target location and the second state or shape being suitable for engaging an inner wall of the target location to anchor the catheter in the target location; and
administering the fluid via the first channel.

32. The method of claim 31, wherein the catheter includes a second distal portion including one or more exit ports communicating with a second channel, the second distal portion distal to the first distal portion, the material comprising a fibrous gel or a fibrous glue injected through the second channel,
wherein, in the first state or shape, the fibrous gel or the fibrous glue behaves as a gel or fluid for traversing the second channel, and
wherein the fibrous gel or the fibrous glue is discharged out of the exit ports of the second distal portion and transitions into the second state or shape in which the fibrous gel or the fibrous glue solidifies into a fibrous bundle for engaging the inner wall of the target location.

33. The method of claim 31, wherein the catheter includes a second distal portion at which the material is applied, the material comprising an adhesive,
wherein, in the first state or shape, the adhesive behaves as a solid without adhesive qualities, and
wherein the adhesive transitions into the second state or shape and behaves with the adhesive qualities after a duration suitable for guiding the catheter to the target location.

34. The method of claim 31, wherein the catheter includes a second distal portion at which the material is attached, the material comprising a flexible super-absorbent material,
wherein, in the first state or shape, the material is contracted around the second distal portion, and
wherein the material absorbs a further fluid to transition into the second state or shape in which the material expands.

35. The method of claim 31, wherein the catheter includes a second distal portion at which the material is attached and further includes suction ports, the catheter including a second channel extending therethrough communicating with the suction ports, the material comprising a sponge,
wherein, in the first state or shape, the sponge is fully contracted around the second distal portion,
wherein, in the second state or shape, the sponge is released from the first state or shape and expands to engage the inner wall of the target location, and
wherein, in a third state or shape, a suction force is applied via the suction ports to distribute the suction force throughout the sponge to partially contract the sponge and the inner wall of the target location.
